(19) United States
(12) Patent Application Publication
(10) Pub. No.: US 2006/0121487 A1
(43) Pub. Date: Jun. 8, 2006

(54) Method for single nucleotide polymorphism detection

(76) Inventors: David Alland, Chatham Township, NJ (US); Manzour Hernando Hazbon, Bloomfield, NJ (US)

Correspondence Address: LICATA & TYRRELL P.C., 66 EAST MAIN STREET, MARLTON, NJ, 08053, US

(21) Appl. No.: 10/540,460

(22) PCT Filed: Dec. 24, 2003
(86) PCT No.: PCT/US03/41136

§ 371 Date: Jan. 17, 2006

Related U.S. Application Data
(60) Provisional application No. 60/437,165, filed on Dec. 27, 2002.

(51) Int. Cl. C12Q 1/68; C12P 19/34

(52) U.S. Cl. 435/6; 435/91.2

(57) ABSTRACT

Method and kits for detecting a single nucleotide polymorphism in an organism via hairpin shaped primers that discriminate between different alleles by situating the 3′ nucleotide at the location of a single nucleotide polymorphism are provided.

### INTRODUCTION

This invention was supported in part by funds from the U.S. government (NIH Grant No. AI46669) and the U.S. government may therefore have certain rights in the invention.

### FIELD OF THE INVENTION

The present invention provides an improved, cost-effective, high-throughput assay useful in single nucleotide polymorphism (SNP) detection. In this method, the amplification refractory mutation (ARMS) assay has been modified to use hairpin shaped primers targeted to the mutation site. This method is extremely useful in efficiently identifying SNPs responsible for drug resistance of infective organisms.

### BACKGROUND OF THE INVENTION

The acquisition of mutations and single nucleotide polymorphisms (SNPs) in target genes is a major cause of the development of drug resistance, variations in immunity, and changes in phenotype of infectious organisms. For example, most causes of drug-resistance in M. tuberculosis appear to be the result of SNPs in particular target genes. However, each SNP occurs at relatively low frequency. Therefore, in the absence of large sequencing studies, it has been difficult to establish statistically valid associations between individual SNPs and resistance to a particular drug. In the case of resistance to the antibiotic isoniazid (INH), only mutations in codon 315 of the katG gene occur with sufficient frequency. The danger of reaching conclusions after analyzing only a limited number of M. tuberculosis isolates is illustrated by the case of SNPs in codon 463 of katG, and codons 269, and 312 of the kasA gene, which were originally identified as resistance associated, but were later shown to be common in INH-susceptible isolates (Abate et al. Eur. J. Clin. Microbiol. Infect. Dis. 2001 20:329-33; Alland et al. J. Bacteriol. 2003 185:3392-9; Lee et al. Antimicrob. Agents Chemother. 1999 43:2087-9; Saint-Joanis et al. Biochem. J. 338 (Pt 3):753-60; Sreevatsan et al. Proc. Natl Acad. Sci. USA 1997 94:9869-74). Accordingly, a comprehensive understanding of the frequency and position of mutations is needed to design genetic assays to detect drug resistance, perform epidemiological investigations identify potential drug targets, identify strains with different immunogenicity, and identify strains with differences in virulence or other phenotype.

SNPs are also a major cause of disease in human and other higher organisms as well as a major determinant of susceptibility to infection. SNPs in certain genes can also determine human response to therapeutic drugs and can also have important effects on susceptibilities to drug related toxicities.

The amplification refractory mutation (ARMS) assay is used routinely to identify SNPs. In this assay two allele-specific primers are designed that are identical except for their 3′ end nucleotide. At the last 3′ nucleotide, one primer is designed to be complementary to the wild-type sequence, while the other primer is complementary to the mutant sequence. Two PCR reactions are performed, one with each of the two 3′ variable primers. Under usual circumstances, the reaction containing the perfectly complementary primer is more efficient than the reaction containing the 3-prime mismatched primer. Thus, the reaction with the complementary primer will contain more PCR product at the end of PCR amplification. This result identifies the correct allele of the target sequence (Newton et al. Nucleic Acids Res. 1989 17:2503-16; Sommer et al. Mayo Clin. Proc. 1989 64:1361-72; Wu et al. Proc. Natl Acad. Sci. USA 1989 86:2757-60).

The ARMS technique can be improved by using real-time PCR (Germer et al. Genome Res. 2000 10:258-66). In real-time PCR, fluorescent techniques measure amplicon synthesis at the annealing or extension segment of each PCR cycle (Bassler et al. Appl. Environ. Microbiol. 1995 61:3724-3728; Livak et al. PCR Methods Appl. 1995 4:357-362). When the fluorescence intensity is plotted at each PCR cycle, successful real-time PCR reactions generate a characteristic rising curve. The point at which the fluorescence of a real-time PCR reaction becomes detectable is referred to as the “threshold cycle”.

In the ARMS method, the well with the perfect primer-template match amplifies more efficiently and has a shorter threshold cycle than the well with the 3′ mismatched primer. Therefore in principle, in this method, the correct allele for each SNP can be determined by comparing the relative threshold cycle values for the each of the paired assay wells.

However, while simple in principle, a number of problems with this method make it less reliable in practice. For example, one problem is that many 3′ mismatches do not destabilize the primer-template hybrid sufficiently enough to delay the cycle threshold, thus making it impossible to assay for SNPs. Another major problem is that PCR amplification of chromosomal DNA under conditions that permit resolution of each SNP is often too inefficient to result in a detectable signal.

In an effort to resolve some of these problems, most 3′ mismatch techniques “preamplify” the target in a nested or hemi-nested protocol. However, nested PCR protocols entail post-PCR manipulations to prepare for the nested reactions.

The present invention provides an improved, cost-effective, high-throughput assay useful in SNP detection.

### SUMMARY OF THE INVENTION

An object of the present invention is to provide a method for detecting single nucleotide polymorphisms in an organism via a modified amplification refractory mutation assay that utilizes a hairpin shaped primer pair that discriminates between different alleles by situating its 3′ nucleotide at the location of a SNP.

Another object of the present invention is to provide an assay kit for detection of a single nucleotide polymorphism comprising a hairpin shaped primer pair that discriminates between different alleles by situating its 3′ nucleotide at the location of a SNP.

### BRIEF DESCRIPTION OF THE FIGURES

FIGS. 1A and 1B provide schematic representations of an assay of the present invention using hairpin (HP) shaped primers. Two reactions with the same DNA target are performed in parallel. FIG. 1A provides a schematic of the HP sequence being fully complementary to the target DNA sequence. FIG. 1B provides a schematic wherein the HP sequence is complementary to the target DNA sequence except for the last 3′ nucleotide of the primer which is complementary to an alternate allelic sequence. As shown in FIG. 1C, the real-time PCR fluorescence curve develops more rapidly in the well where the HP sequence is fully complementary to the target DNA sequence (earlier Ct), indicating the presence of allele A.

FIG. 2A through 2D provide a comparison of HP (FIG. 2A), linear primer (LP; FIG. 2B), linearized-HP tail (LHP; FIG. 2C) and substituted extended-LHP tail (ELHP; FIG. 2D) with and without secondary mutations. Ct for each paired reactions are shown as a single point. The X axis denotes the Ct of the reaction with allele “A” primers and the Y axis denotes the Ct of the reaction with allele “B” primers. Values above the diagonal line (marking the place where Ct(B)/Ct(A)>1) indicate that an A allele SNP should be present, values below the diagonal indicate that a B allele SNP should be present. Primers with secondary mutations are represented by diamonds, and primers without secondary mutations are represented by triangles. Closed symbols correspond to successful assays; open symbols indicate erroneous or indeterminate (ΔCt<5) assays.

FIG. 3A-3C show the primers (FIG. 3A), their secondary structure (FIG. 3B) and results (FIG. 3C) of detecting four alleles at a single codon using the HP assay. Assays were designed to detect mutations S315I (AGC→ATC), S315N (AGC→AAC) and S315T (AGC→ACC) in the katG gene. FIG. 3A shows the DNA sequence of the target (SEQ ID NO:120) and HP primers FHP katG^{S315T} (SEQ ID NO:121), FHP katG^{S315N} (SEQ ID NO:122), FHP katG^{S15I} (SEQ ID NO:123) and FHP katG^{S315} (SEQ ID NO:124). Secondary mutations and SNPs are shown in bold capitals and 5′-end tails are underlined. The location of the constant primer is indicated by lower-case bolding in the target (SEQ ID NO:120). FIG. 3B shows predicted secondary structures for each of the HP primers at 60° C., 2 mM MgCl₂ (annealing conditions). FIG. 3C shows real time PCR results using chromosomal DNA from H37Rv as WT control(katG315; filled square), and the MUT isolates I-524 (katGS315I; open diamond), M-5036 (katGS315N; filled circle) and M-5153 (katGS315T; filled triangle). An earlier Ct was observed for each matched reaction, indicating the correct allele.

### DETAILED DESCRIPTION OF THE INVENTION

The present invention provides low-cost, high-throughput methods and assay kits for detection of single nucleotide polymorphisms (SNPs) in an organism. The methods and kits of the present invention enable analysis of a large number of isolates, thereby providing a means for comprehensive understanding of the frequency and position of mutations in an organism. In the present invention, the amplification refractory mutation or ARMS assay has been modified to utilize hairpin shaped primers. The hairpin shaped primers used in the methods and assay kits of the present invention discriminate between different alleles by situating their 3′ nucleotide at the location of the SNP.

For purposes of the present invention, by the term organism it is meant to be inclusive of any organism with nucleic acid sequences including viruses, prokaryotes, and eukaryotes, including humans.

By hairpin shaped primer, as used herein, it is meant an oligonucleotide with a 5′ tail that hybridizes to its 3′ end. Accordingly, hairpin primers used in the present invention form a stem-and-loop structure. In a preferred embodiment, the hairpin shaped primers have a melting point (Tm) of about 5° C. to about 10° C. above the Tm of the primer-target hybrid. However, hairpin shaped primers with tails that exhibit a Tm outside of this preferred range may also be used. The length or size of the primer is selected based upon the desired annealing temperature in the PCR reaction. A preferred size of the primer is about 15 to about 25 bases. In general, the primers are comprised of DNA. However RNA, PNA, and modified nucleotides can also be used in the primers of the present invention.

Use of hairpin shaped primers in the method of the present invention decreases non-specific primer target hybridizations observed with the traditional ARMS assay during the early stages of PCR, thus greatly simplifying assay design and interpretation. Furthermore, it has recently been reported that hairpin primers are less likely to form primer dimers in PCR (Nazarenko et al. Nucleic Acids Res. 2002 30(9):e37). In addition, in contrast to Taqman probes and molecular beacons, the hairpin shaped primers used in the present invention do not require fluorophore labeling for detection in simple reactions thereby dramatically lowering assay costs. Hairpin shaped primers of the present invention can be labeled with a fluorophore, however, to permit multiplexed reactions monitored by real-time PCR.

To produce hairpin shaped primers for use in the SNP detection methods and assay kits of the present invention, pairs of primers that varied at the last 3′ nucleotide were first prepared. As in standard 3′ primer mismatch PCR, one primer was designed to be complementary to the wild-type sequence, while the other primer was designed to be complementary to the mutant sequence. Additional modifications were then made at the 5′ end of each primer so that it was reverse-complementary to the first 5-8 nucleotides of its 3′ end, conferring to it the ability to form a “hairpin”. The primers were designed so that the melting temperature of the hairpin stems was approximately six degrees above the melting temperature of the primers to their complementary targets. Additional mutations were occasionally necessary in the “loop” of the primers to achieve the desired secondary structure and melting temperature. These primers formed a uni-molecular stem-and-loop structure in solution, but hybridized to complementary DNA targets to form linear bimolecular complexes below the melting temperature of the primers to their targets. It was found that this type of “hairpin” primer configuration greatly decreased the amplification efficiency of targets that were mismatched for a SNP at the 3′ nucleotide of the primer. This is believed to be due to the propensity of a hairpin primer to disassociate from a mismatched target in preference to forming a stable uni-molecular stem-and-loop structure. In contrast, hairpin primers that were perfectly matched to their targets formed more stable bimolecular primer-target hybrids. This enabled perfectly matched hairpin primers to prime PCR reactions with amplification efficiencies similar to linear PCR primers. Mismatched hairpin primers usually delayed amplification by 5 to 18 PCR cycles. To increase amplification efficiency, the size of the amplicons was substantially reduced from typical size of approximately 100 to 300 bases. Most assays were designed to produce amplicons that are only a few base pairs longer than the sum of both PCR primers. By combining hairpin primers with these shorter amplicons, the success rate of each newly designed SNP assay has been dramatically improved.

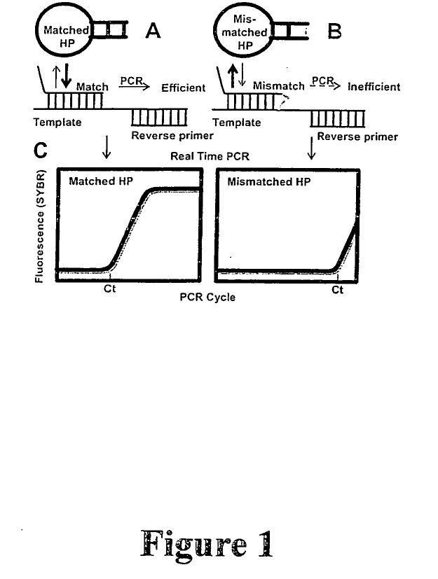

Assays using the hairpin shaped primers are very easy to perform and analyze. A schematics of setting forth the principles of the assay are depicted in FIGS. 1A and 1B.

In a preferred embodiment, real-time PCR techniques are used for monitoring. For example, for a high-throughput screening assay, primer pairs (constant and hairpin shaped primer) can be added and dried in 384-well plates. By constant primer it is meant a linear or hairpin reverse primer that is conserved in both SNP alleles. The appropriate reaction mixes containing SYBR green dye, dNTPs, PCR buffer, water, MgCl₂ and Taq polymerase, optimized for the mutation are then added to the primer-containing wells, the plate is sealed, and real-time PCR is performed, for example, on an ABI 7900 real-time PCR machine or other real-time PCR instrument for 30 to 50 thermal-cycles. A touchdown protocol is usually incorporated as well. The threshold cycle is automatically calculated and the correct SNP allele is identified by comparing the threshold cycle of each paired well. The results can also be examined graphically to identify problematic results.

The specificity of real-time PCR reactions in this embodiment is high because all reactions are performed in sealed plates. These plates are never opened, therefore false positive results caused by cross-contamination of amplicons do not occur.

In another assay embodiment, PCR is performed without real-time monitoring and amplicon production is measured at the completion of the PCR reaction.

The assays of the present invention can be performed at relatively low cost as compared to other methods for SNP detection. Amplicon generation by the method of the present invention can be detected by adding inexpensive SYBR green dye to the PCR mix. Accordingly, the assay does not require use of expensive fluorescent-labeled primers or probes. Costs are further decreased because reaction volumes can be as low as four microliters. In addition, the 384-well format of real-time PCR machines such as the ABI 7900 enables very high throughput.

Further, as will be understood by one of skill in the art upon reading the instant application, the SNP detection assay of the present invention can be routinely adapted for use of other amplification methods including, but not limited to, TMA and SDA.

The present invention also provides assay kits for detecting a single nucleotide polymorphism in an organism. Kits of the present invention comprise a hairpin shaped primer that discriminates between different alleles by situating its 3′ nucleotide at the location of a single nucleotide polymorphism. Kits also preferably comprise additional ingredients for use in the amplification method to be used as well as detection of the generated amplicons. For example, for amplification by PCR, the assay kits of the present invention may further comprise SYBR green dye and components for the PCR mixture, as well as additional primers labeled with a fluorophore.

The ability of the method of the present invention to detect SNPs rapidly and with high sensitivity was demonstrated in Mycobacterium tuberculosis and other organisms. In these experiments, amplification refractory mutation (ARMS) SNP assays were modified by converting the SNP-detecting linear primers (LPs) in the ARMS assay to hairpin-shaped primers (HPs) through the addition of a 5′ tail complementary to the 3′-end of the LP. The improved ability of these primers to detect SNPs in M. tuberculosis was compared in a real time PCR reaction using SYBR-I green dye. LPs resulted in incorrect or indeterminate allele designation for six of the thirteen SNP alleles tested in seven different SNP assays, while HPs determined the correct SNP in all cases. The cycle threshold differences (ΔCt) were also compared between the reactions containing primer-template matches and the reactions containing primer-template mismatches (where a larger ΔCt indicates a more robust assay). The use of HPs dramatically improved the mean ΔCt values for the SNP assays (7.6 for LPs and 11.2 for HPs).

Further, ninety-eight different HP assays were designed for SNPs previously associated with resistance to the antibiotic isoniazid to test the large-scale utility of the HP approach. Assay design was successful in 72.4%, 83.7%, 88.8% and 92.9% of the assays after one to four rounds of assay design respectively. Thus, the HP SNP assays of the present invention provide a simple, sensitive, robust, and inexpensive technique for SNP detection.

The following nonlimiting examples are provided to further illustrate the present invention.

### EXAMPLE

### Example 1

### M. tuberculosis Isolates and Chromosomal DNA Extraction

Chromosomal DNA was extracted using the CTAB method as described by van Embden et al. (J. Clin. Microbiol. 1993 31:406-9). The reference strain H37Rv was used as a wild-type (WT) control. Clinical isolates I-524, M-5036 and M-5455 were used as mutant (MUT) controls for katGS315I, katGS315N and katGS315T, respectively. For the remaining SNPs tested, the isolates used are indicated in Table 1.

TABLE 1
Sequences of Primers Used

| | | | | | | Amp. | |
|---|---|---|---|---|---|---|---|
| | | | | | | 1 ng | MUT |
| SNP | LP | HP | LHP | ELHP | Constant Primer | (bp)² | Isolate |
| katG315¹ | catacgTcctcgatgccgc | GCGGC | CGCCC | ATATACGCCC | ccggtaaggacgcgatcac | 40 | M-5455 |
| | (SEQ ID NO: 1) | (SEQ ID NO: 29) | (SEQ ID NO: 57) | (SEQ ID NO: 85) | (SEQ ID NO: 113) | | |
| katGS315T¹ | catacgTcctcgatgccgG | CCGGC | CGCCC | ATATACGCCC | | | |
| | (SEQ ID NO: 2) | (SEQ ID NO: 30) | (SEQ ID NO: 58) | (SEQ ID NO: 86) | | | |
| katG315¹ | catacgacctcgatgccgc | GCGGC | CGCCC | ATATACGCCC | | | |
| | (SEQ ID NO: 3) | (SEQ ID NO: 31) | (SEQ ID NO: 59) | (SEQ ID NO: 87) | | | |
| katGS315T¹ | catacgacctcgatgccgG | CCGGC | CGCCC | ATATACGCCC | | | |
| | (SEQ ID NO: 4) | (SEQ ID NO: 32) | (SEQ ID NO: 60) | (SEQ ID NO: 88) | | | |
| katG485 | cgtcCtcgttccgtgg | CCACGG | GCTCGC | GATCGCGCTCGC | aggcggatgcgacca | 58 | N/A³ |
| | (SEQ ID NO: 5) | (SEQ ID NO: 33) | (SEQ ID NO: 61) | (SEQ ID NO: 89) | (SEQ ID NO: 114) | | |
| katGG485V | cgtcCtcgttccgtgT | ACACGG | TCTCGC | GATCGCTCTCGC | | | |
| | (SEQ ID NO: 6) | (SEQ ID NO: 34) | (SEQ ID NO: 62) | (SEQ ID NO: 90) | | | |
| katG485 | cgtcgtcgttccgtgg | CCACGG | GCTCGC | GATCGCGCTCGC | | | |
| | (SEQ ID NO: 7) | (SEQ ID NO: 35) | (SEQ ID NO: 63) | (SEQ ID NO: 91) | | | |
| katGG485V | cgtcgtcgttccgtgT | ACACGG | TCTCGC | GATCGCTCTCGC | | | |
| | (SEQ ID NO: 8) | (SEQ ID NO: 36) | (SEQ ID NO: 64) | (SEQ ID NO: 92) | | | |
| kasA269 | cgattCctgggtgccg | CGGCA | GCGGA | GCGGAGCGGA | | 36 | M-5279 |
| | (SEQ ID NO: 9) | (SEQ ID NO: 37) | (SEQ ID NO: 65) | (SEQ ID NO: 93) | | | |
| kasAG269S | cgattCctgggtgccA | TGGCA | ACGGA | GCGGAACGGA | aaaggcgtccgaggtgatac | | |
| | (SEQ ID NO: 10) | (SEQ ID NO: 38) | (SEQ ID NO: 66) | (SEQ ID NO: 94) | (SEQ ID NO: 115) | | |
| kasA269 | cgattgctgggtgccg | CGGCA | GCGGA | GCGGAGCGGA | | | |
| | (SEQ ID NO11) | (SEQ ID NO: 39) | (SEQ ID NO: 67) | (SEQ ID NO: 95) | | | |
| kasAG269S | cgattgctgggtgccA | TGGCA | ACGGA | GCGGAACGGA | | | |
| | (SEQ ID NO: 12) | (SEQ ID NO: 40) | (SEQ ID NO: 68) | (SEQ ID NO: 95) | | | |
| inhA21 | atcatcTccgactcgtcgat | ATCGACG | ATGCACC | ACGAGCAAATGCACC | gctacccgtgcgatgtgaa | 39 | M-5502 |
| | (SEQ ID NO: 13) | (SEQ ID NO: 41) | (SEQ ID NO: 69) | (SEQ ID NO: 97) | (SEQ ID NO: 116) | | |
| inhAI21T | atcatcTccgactcgtcgaC | GTCGACG | CTGCACC | ACGAGCAACTGCACC | | | |
| | (SEQ ID NO: 14) | (SEQ ID NO: 42) | (SEQ ID NO: 70) | (SEQ ID NO: 98) | | | |
| inhA21 | aatcatcaccgactcgtcgat | ATCGACG | ATGCACC | ACGAGCAAATGCACC | | | |
| | (SEQ ID NO: 15) | (SEQ ID NO: 43) | (SEQ ID NO: 71) | (SEQ ID NO: 99) | | | |
| inhAI21T | aatcatcaccgactcgtcgaC | GTCGACG | CTGCACC | ACGAGCAACTGCACC | | | |
| | (SEQ ID NO: 16) | (SEQ ID NO: 44) | (SEQ ID NO: 72) | (SEQ ID NO: 100) | | | |
| inhA94 | ggcaAgaacccaatcga | TCGATTGGA | TGGAATGCA | GAGGCAAGTGGAATGCA | cgggcaacaagctcgac | 46 | M-5041 |
| | (SEQ ID NO: 17) | (SEQ ID NO: 45) | (SEQ ID NO: 73) | (SEQ ID NO: 101) | (SEQ ID NO: 117) | | |
| inhAS94A | ggcaAgaacccaatcgC | GCGATTGGA | CGGAATGCA | GAGGCAAGCGGAATGCA | | | |
| | (SEQ ID NO: 18) | (SEQ ID NO: 46) | (SEQ ID NO: 74) | (SEQ ID NO: 102) | | | |
| inhA94 | ggcatgaacccaatcga | TCGATTGGA | TGGAATGCA | GAGGCAAGTGGAATGCA | | | |
| | (SEQ ID NO: 19) | (SEQ ID NO: 47) | (SEQ ID NO: 75) | (SEQ ID NO: 103) | | | |
| iuhAS94A | ggcatgaacccaatcgC | GCGATTGGA | CGGAATGCA | GAGGCAAGCGGAATGCA | | | |
| | (SEQ ID NO: 20) | (SEQ ID NO: 48) | (SEQ ID NO: 76) | (SEQ ID NO: 104) | | | |
| ahpC73 | gcCgtcctcgaactcgtc | GACGAGT | CACCAGA | TGCATGTCACCAGA | | 38 | M-5167 |
| | (SEQ ID NO: 21) | (SEQ ID NO: 49) | (SEQ ID NO: 77) | (SEQ ID NO: 105) | | | |
| ahpCD73H | gcCgtcctcgaactcgtG | CACGAGT | GACCAGA | TGCATGTGACCAGA | cggcgttcagcaagctc | | |
| | (SEQ ID NO: 22) | (SEQ ID NO: 50) | (SEQ ID NO: 78) | (SEQ ID NO: 106) | (SEQ ID NO: 118) | | |
| ahpC73 | gcggtcctcgaactcgtc | GACGAGT | CACCAGA | TGCATGTCACCAGA | | | |
| | (SEQ ID NO: 23) | (SEQ ID NO: 51) | (SEQ ID NO: 79) | (SEQ ID NO: 107) | | | |
| ahpCD73H | gcggtcctcgaactcgtG | CACGAGT | GACCAGA | TGCATGTGACCAGA | | | |
| | (SEQ ID NO: 24) | (SEQ ID NO: 52) | (SEQ ID NO: 80) | (SEQ ID NO: 108) | | | |
| ndh268 | ggcTccgtccggcg | CGCCGA | GGCGCA | TACGAGGGCGCA | cagaccttgcaggccgactc | 38 | I-591 |
| | (SEQ ID NO: 25) | (SEQ ID NO: 53) | (SEQ ID NO: 81) | (SEQ ID NO: 109) | (SEQ ID NO: 119) | | |
| ndhR268H | ggcTccgtccggcA | TGCCGA | AGCGCA | TACGAGAGCGCA | | | |
| | (SEQ ID NO: 26) | (SEQ ID NO: 54) | (SEQ ID NO: 82) | (SEQ ID NO: 110) | | | |
| ndh268 | ggcaccgtccggcg | CGCCGA | GGCGCA | TACGAGGGCGCA | | | |
| | (SEQ ID NO: 27) | (SEQ ID NO: 55) | (SEQ ID NO: 83) | (SEQ ID NO: 111) | | | |
| ndhR268H | ggcaccgtccggcA | TGCCGA | AGCGCA | TACGAGAGCGCA | | | |
| | (SEQ ID NO: 28) | (SEQ ID NO: 56) | (SEQ ID NO: 84) | (SEQ ID NO: 112) | | | |

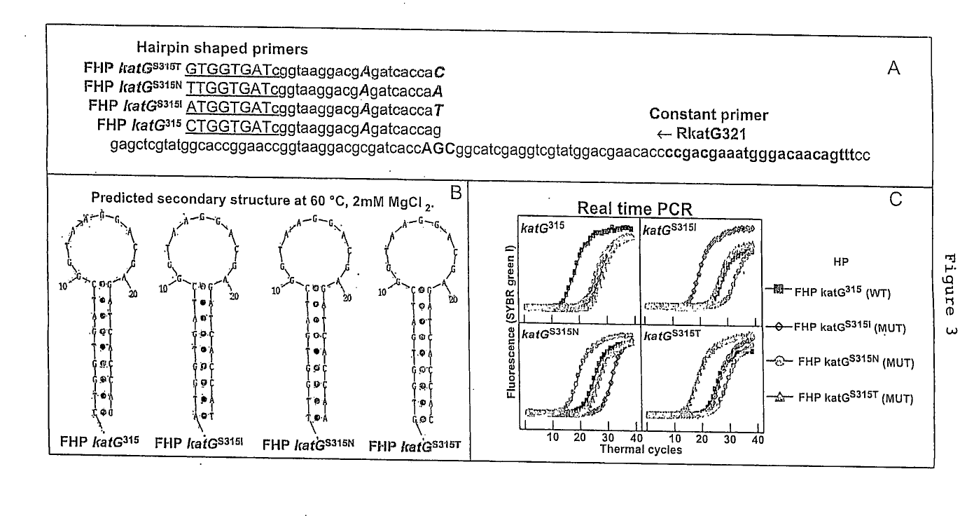

Shown (left to right): linear primer (LP) sequence, additional HP tail (HP), substituted linearized-HP (LHP) tail, substituted extended-LHP (ELHP) tail. Mutations (either SNPs or secondary mutations) are shown underlined in bold capitals
¹These primers detect the SNPs as depicted in FIG. 3 but using a complementary strand.
²Base-pairs (5′-end tail sequences not included)
³Not available

### Example 2

### Real Time PCR

All PCR reactions were performed in an Applied Biosystems 7900HT sequence detector system with the 384-well block for real-time PCR. Thermal conditions were as follows: Stage 1: 95° C. 10 minutes, 70° C. for 30 seconds; Stage 2: 72° C. for 30 seconds, 95° C. 20 seconds, 69° C. for 30 seconds lowering one degree in the last step for every cycle during 10 cycles; and Stage 3: 72° C. for 30 seconds, 95° C. 20 seconds, 60° C. for 30 seconds, repeated 40 times. Data was collected in the last step of stage 3 for analysis with the SDS software version 2.0a23 (Applied Biosystems, CA). Every well was loaded with PCR cocktail containing: 1×Amplitaq Gold polymerase buffer, 0.15 U of Amplitaq Gold polymerase (Perkin-Elmer, CA), 2 mM MgCl₂, 2.5 pmol of each primer, 1×SYBR green I (Molecular Probes Inc., OR), 1.75 ng ROX [6-carboxy-X-rhodamine, succinimidyl ester (6-ROX, SE)] (Molecular Probes Inc., OR) (used as a reference dye), and either 0.1 ng of chromosomal DNA, or 10⁵ molecules of the artificial template, or an equal volume of water (no DNA control), followed by sufficient water to result in a final volume of 5 μl.

### Example 3

### HP-assay Design

Primers (Invitrogen, CA, or Illumina, CA), whose sequences are shown in Table 1, were designed using the Primer Express Software version 2.0 (Applied Biosystems, CA) to produce short amplicons (30-90 base pairs long), and to anneal between 60-65° C. A tail was added to the 5′-end of the SNP-detecting primer in order to produce a stem with the 3′-end of the primer. The stem was designed using mfold software (see bioinfo.rpi.edu/applications/mfold/old/dna/ of the world wide web) to have a Tm of 67-70° C. with a free energy ΔG) between −0.5 and −2.0. Two single-stranded artificial templates (WT and MUT) were designed to test the discriminatory power of each primer set, and chromosomal DNA of M. tuberculosis H37Rv was used as a WT control.

### Example 4

### HP High Throughput Assay

384-well plates (Applied Biosystems, CA) were loaded with 5 μl per well of the SNP specific and constant primer mix using a Biomek 2000 Laboratory Automation Workstation (Beckman Coulter, CA). Plates were completely dried overnight inside a laminar flow cabinet, and kept in air-tight plastic bags at −20° C. until used. Five μl/well of the PCR cocktail containing all the components except the primers were then loaded into the microtiter plates. The plates were vortexed and then centrifuged prior to being loaded into the robot of the sequence detector system apparatus.

### Example 5

### Comparison of Linear Primers (LP) and HP

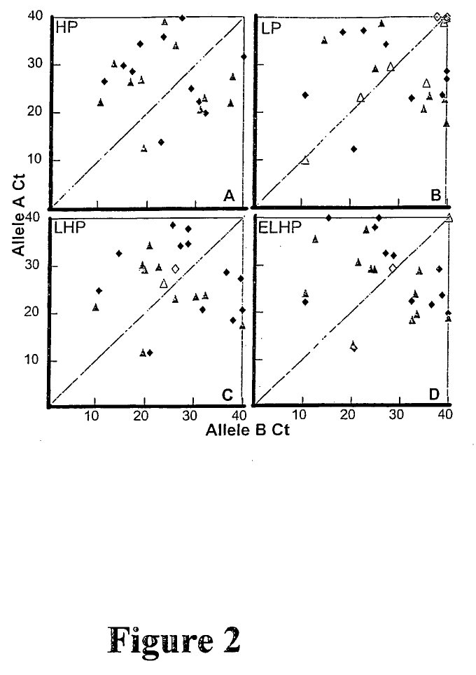

The ability of LPs and HPs to distinguish between two or more SNP alleles in seven different SNP assays was compared. The principles of the HP assay are set forth in FIGS. 1A and 1B. For this comparison, sets of LPs for standard ARMS assays were first designed using Primer Express software. Then, a second set of primers was designed identical to the first set except that the SNP-specific primer was modified to form a stem-and-loop structure as described above. Assays containing the conventional LPs and otherwise identical assays containing the HPs were then tested for their ability distinguish between M. tuberculosis SNPs. Seven actual drug resistance-associated SNPs present in M. tuberculosis were tested, using M. tuberculosis chromosomal DNA to ensure that the results would be applicable in the subsequent investigations. Each assay was performed in quadruplicate and the average Ct values for each quadruplicate were calculated. Corresponding LP and HP assays were compared on two characteristics: 1) the ability to designate the correct SNP (versus an incorrect or indeterminate assignment), and 2) the average cycle threshold difference (ΔCt) between the reactions containing primer-template matches and the reactions containing primer-template mismatches (where a larger ΔCt indicates a more robust assay). Assays were considered indeterminate if the ΔCt was lower than five. The HP assay was found to have a much greater discriminatory power than the LP assay. The LP assay identified the correct SNP in only 7 of 13 assays (producing one incorrect and five indeterminate results), while the HP assay identified the correct SNP in all 13 assays, demonstrating the superiority of this format (see FIGS. 2A and 2B, triangles). Furthermore, the HP assay appeared to be more sensitive for SNPs as the ΔCt values were greater in the HP assays compared to the LP assays (See Table 2).

TABLE 2
Ct averages of 7 SNP assays using different primer sets

| | | | | Rejected |
|---|---|---|---|---|
| Primer set | Match² Ct | Mismatch³ Ct | ΔCt⁴ | assays⁵ |
| LP | 25.0 | 32.6 | 7.6 | 6 |
| HP | 19.6 | 30.8 | 11.2 | 0 |
| LHP | 19.6 | 29.1 | 9.5 | 1 |
| ELHP | 21.1 | 32.1 | 11.0 | 1 |

¹See Example 1 for description of isolates used
²Match reaction: PCR reaction in which the 3′end of the primer is complementary to the target
³Mismatch reaction: PCR reaction in which the 3′end of the primer is not complementary to the target
⁴Average of mismatch Ct − average of matched Ct
⁵Out of total of 13 assays performed

### Example 6

### HP Comparison With Linearized HP (LHP) and Extended-LHP (ELHP)

To investigate the possibility that the enhanced ability of the HP configuration might be due to the extended 5′ tail of the SNP-specific primer rather than to the actual stem-and-loop, “linearized” HPs (LHPs) were created by mutating the 5′ tail of each HP primer so that it could no longer form a stem with the 3′ end. The LHPs were designed so that no new secondary structures were formed and so that they had the same GC content as the HPs. Each SNP assay was repeated with LHP sets. The performance of the LHP assays was intermediate between that of the linear primers and HP primers (see FIG. 2C). LHP assays only produced one indeterminate result, however the ΔCt values averaged 1.7 cycles less than the HP assays (Table 2). To determine if further lengthening of the 5′ tail would continue to enhance the ability of LHPs to distinguish among SNPs, a corresponding set of “extended” LHPs (ELHPs) was created by doubling the length of each 5′ tail. The performance of assays with ELHPs was compared with the other primers. It was found that ELHP assays produced results that were similar to LHP assays, with one indeterminate result out of 13 (see FIG. 2D, triangles). Notably, assays with ELHPs had improved ΔCt values, with an average ΔCt that was equivalent to the average ΔCt of reactions containing HPs (Table 2). However, the ΔCt values of individual HP assays fell within a more narrow range than the ΔCts of the ELHPs, thus indicating that the HP-based assays remained superior.

### Example 7

### Evaluation of the Insertion of a Secondary Mismatch

The effect of inserting additional base pair mismatches into the HPs was tested. A secondary mismatch was inserted into each primer either towards the center of the loop or at the end of the stem. Mismatches were designed so that total GC content was maintained. The results indicated that the secondary mismatches resulted in later Cts in both match and mismatch reactions, and led to ΔCt values that were improved by 1.1, 0.2, 2.1 and 0.8 cycles for LP, HP, LHP and ELHP respectively (FIG. 2, diamonds). The number of rejected assays did not vary with the secondary mismatch, except for LPs where only four reactions were rejected (one incorrect and 3 indeterminate results). The incorporation of a secondary mismatch can also play an important role during the HP design. The mismatch confers flexibility to the design process and can be used to avoid undesired secondary structures.

### Example 8

### The HP Approach for Loci Containing Multiple Alleles

A single codon can contain more than two SNP alleles. This is the case for position 315 in the katG gene of M. tuberculosis, which is the most common position mutated in INH resistant clinical isolates (Ramaswamy and Musser Tuber. Lung Dis. 1998 79:3-29). The ability of the HP assay to test for four possible alleles at this position was investigated. A single WT HP primer and three different MUT HP primers were designed to be complementary to each katG315 allele. The HPs were then tested in assays with chromosomal M. tuberculosis DNA containing each mutation. The results show that the HP assay can easily distinguish among all four alleles (FIG. 3).

### Example 9

### Success Rate of Large-scale HP Assay Design

HP assays were designed for 207 different M. tuberculosis SNPs at 98 different polymorphic sites previously associated with resistance to INH to test the utility of the HP approach in large-scale SNP analysis. Each assay was tested on chromosomal DNA from M. tuberculosis H37Rv (WT control) and both artificial templates. MUT chromosomal DNA was also used when available. Ninety-one functional HP assays (most of which included a secondary mutation) were successfully designed that detected SNPs in the katG, kasA, ahpc, inhA, mabA and ndh genes of M. tuberculosis. Assays that detected insertions and deletions were developed using the same parameters. Design success rates as a function of number of design attempts are shown in Table 3.

TABLE 3
Success rate of HP assay design

| Attempt¹ | Success rate² |
|---|---|
| 1 | 72.4% (71/98) |
| 2 | 83.7% (82/98) |
| 3 | 88.8% (87/98) |
| 4 | 92.9% (91/98) |

¹Number of times a HP assay was redesigned until discriminatory
²Percentage of HP assays able to discriminate amongst SNP alleles according to the predefined criteria at a particular design round

The sensitivity of the assays in terms of the amount of chromosomal DNA required was also examined. Most assays gave consistent results with less than 0.05 ng/well of chromosomal DNA. Preferably 0.1 ng/well of chromosomal DNA is used as this amount resulted in smoother amplification curves.

What is claimed is:

1. An assay for detecting a single nucleotide polymorphism in an organism comprising:
amplifying a 30 to 90 base pair nucleic acid molecule of an organism using a hairpin shaped primer that discriminates between different alleles by situating its 3′ nucleotide at the location of a single nucleotide polymorphism; and
measuring threshold cycle or amplification efficiency or amount of amplified product wherein a lower amplification efficiency or delayed threshold cycle or a difference in the amount of amplified product is indicative of a mismatch between the primer and the organism and a single nucleotide polymorphism in the organism.

2. The assay of claim 1 wherein the nucleic acid sequence of the organism is amplified by PCR.

3. The assay of claim 2 wherein the PCR performed is real-time PCR.

4. The assay of claim 2 wherein amplicon production is measured at the completion of the PCR reaction.

5. The assay of claim 1 wherein the hairpin shaped primer comprises DNA.

6. The assay of claim 1 wherein the hairpin shaped primer comprises RNA.

7. The assay of claim 1 wherein the hairpin shaped primer comprises PNA.

8. An assay kit for detecting a single nucleotide polymorphism in an organism comprising a hairpin shaped primer for amplifying a 30 to 90 base pair nucleic acid molecule, wherein the hairpin shaped primer discriminates between different alleles by situating its 3′ nucleotide at the location of a single nucleotide polymorphism.

9. The assay kit of claim 8 wherein the hairpin shaped primer comprises DNA.

10. The assay kit of claim 8 wherein the hairpin shaped primer comprises RNA.

11. The assay kit of claim 8 wherein the hairpin shaped primer comprises PNA.


SEQUENCE LISTING (124 sequences)

SEQ ID NO: 1 - DNA, 19 bp, Artificial sequence
  Synthetic
catacgtcctcgatgccgc

SEQ ID NO: 2 - DNA, 19 bp, Artificial sequence
  Synthetic
catacgtcctcgatgccgg

SEQ ID NO: 3 - DNA, 19 bp, Artificial sequence
  Synthetic
catacgacctcgatgccgc

SEQ ID NO: 4 - DNA, 19 bp, Artificial sequence
  Synthetic
catacgacctcgatgccgg

SEQ ID NO: 5 - DNA, 16 bp, Artificial sequence
  Synthetic
cgtcctcgttccgtgg

SEQ ID NO: 6 - DNA, 16 bp, Artificial sequence
  Synthetic
cgtcctcgttccgtgt

SEQ ID NO: 7 - DNA, 16 bp, Artificial sequence
  Synthetic
cgtcgtcgttccgtgg

SEQ ID NO: 8 - DNA, 16 bp, Artificial sequence
  Synthetic
cgtcgtcgttccgtgt

SEQ ID NO: 9 - DNA, 16 bp, Artificial sequence
  Synthetic
cgattcctgggtgccg

SEQ ID NO: 10 - DNA, 16 bp, Artificial sequence
  Synthetic
cgattcctgggtgcca

SEQ ID NO: 11 - DNA, 16 bp, Artificial sequence
  Synthetic
cgattgctgggtgccg

SEQ ID NO: 12 - DNA, 16 bp, Artificial sequence
  Synthetic
cgattgctgggtgcca

SEQ ID NO: 13 - DNA, 20 bp, Artificial sequence
  Synthetic
atcatctccgactcgtcgat

SEQ ID NO: 14 - DNA, 20 bp, Artificial sequence
  Synthetic
atcatctccgactcgtcgac

SEQ ID NO: 15 - DNA, 21 bp, Artificial sequence
  Synthetic
aatcatcaccgactcgtcgat

SEQ ID NO: 16 - DNA, 21 bp, Artificial sequence
  Synthetic
aatcatcaccgactcgtcgac

SEQ ID NO: 17 - DNA, 17 bp, Artificial sequence
  Synthetic
ggcaagaacccaatcga

SEQ ID NO: 18 - DNA, 17 bp, Artificial sequence
  Synthetic
ggcaagaacccaatcgc

SEQ ID NO: 19 - DNA, 17 bp, Artificial sequence
  Synthetic
ggcatgaacccaatcga

SEQ ID NO: 20 - DNA, 17 bp, Artificial sequence
  Synthetic
ggcatgaacccaatcgc

SEQ ID NO: 21 - DNA, 18 bp, Artificial sequence
  Synthetic
gccgtcctcgaactcgtc

SEQ ID NO: 22 - DNA, 18 bp, Artificial sequence
  Synthetic
gccgtcctcgaactcgtg

SEQ ID NO: 23 - DNA, 18 bp, Artificial sequence
  Synthetic
gcggtcctcgaactcgtc

SEQ ID NO: 24 - DNA, 18 bp, Artificial sequence
  Synthetic
gcggtcctcgaactcgtg

SEQ ID NO: 25 - DNA, 14 bp, Artificial sequence
  Synthetic
ggctccgtccggcg

SEQ ID NO: 26 - DNA, 14 bp, Artificial sequence
  Synthetic
ggctccgtccggca

SEQ ID NO: 27 - DNA, 14 bp, Artificial sequence
  Synthetic
ggcaccgtccggcg

SEQ ID NO: 28 - DNA, 14 bp, Artificial sequence
  Synthetic
ggcaccgtccggca

SEQ ID NO: 29 - DNA, 5 bp, Artificial sequence
  Synthetic
gcggc

SEQ ID NO: 30 - DNA, 5 bp, Artificial sequence
  Synthetic
ccggc

SEQ ID NO: 31 - DNA, 5 bp, Artificial sequence
  Synthetic
gcggc

SEQ ID NO: 32 - DNA, 5 bp, Artificial sequence
  Synthetic
ccggc

SEQ ID NO: 33 - DNA, 6 bp, Artificial sequence
  Synthetic
ccacgg

SEQ ID NO: 34 - DNA, 6 bp, Artificial sequence
  Synthetic
acacgg

SEQ ID NO: 35 - DNA, 6 bp, Artificial sequence
  Synthetic
ccacgg

SEQ ID NO: 36 - DNA, 6 bp, Artificial sequence
  Synthetic
acacgg

SEQ ID NO: 37 - DNA, 5 bp, Artificial sequence
  Synthetic
cggca

SEQ ID NO: 38 - DNA, 5 bp, Artificial sequence
  Synthetic
tggca

SEQ ID NO: 39 - DNA, 5 bp, Artificial sequence
  Synthetic
cggca

SEQ ID NO: 40 - DNA, 5 bp, Artificial sequence
  Synthetic
tggca

SEQ ID NO: 41 - DNA, 7 bp, Artificial sequence
  Synthetic
atcgacg

SEQ ID NO: 42 - DNA, 7 bp, Artificial sequence
  Synthetic
gtcgacg

SEQ ID NO: 43 - DNA, 7 bp, Artificial sequence
  Synthetic
atcgacg

SEQ ID NO: 44 - DNA, 7 bp, Artificial sequence
  Synthetic
gtcgacg

SEQ ID NO: 45 - DNA, 9 bp, Artificial sequence
  Synthetic
tcgattgga

SEQ ID NO: 46 - DNA, 9 bp, Artificial sequence
  Synthetic
gcgattgga

SEQ ID NO: 47 - DNA, 9 bp, Artificial sequence
  Synthetic
tcgattgga

SEQ ID NO: 48 - DNA, 9 bp, Artificial sequence
  Synthetic
gcgattgga

SEQ ID NO: 49 - DNA, 7 bp, Artificial sequence
  Synthetic
gacgagt

SEQ ID NO: 50 - DNA, 7 bp, Artificial sequence
  Synthetic
cacgagt

SEQ ID NO: 51 - DNA, 7 bp, Artificial sequence
  Synthetic
gacgagt

SEQ ID NO: 52 - DNA, 7 bp, Artificial sequence
  Synthetic
cacgagt

SEQ ID NO: 53 - DNA, 6 bp, Artificial sequence
  Synthetic
cgccga

SEQ ID NO: 54 - DNA, 6 bp, Artificial sequence
  Synthetic
tgccga

SEQ ID NO: 55 - DNA, 6 bp, Artificial sequence
  Synthetic
cgccga

SEQ ID NO: 56 - DNA, 6 bp, Artificial sequence
  Synthetic
tgccga

SEQ ID NO: 57 - DNA, 5 bp, Artificial sequence
  Synthetic
cgccc

SEQ ID NO: 58 - DNA, 5 bp, Artificial sequence
  Synthetic
cgccc

SEQ ID NO: 59 - DNA, 5 bp, Artificial sequence
  Synthetic
cgccc

SEQ ID NO: 60 - DNA, 5 bp, Artificial sequence
  Synthetic
cgccc

SEQ ID NO: 61 - DNA, 6 bp, Artificial sequence
  Synthetic
gctcgc

SEQ ID NO: 62 - DNA, 6 bp, Artificial sequence
  Synthetic
tctcgc

SEQ ID NO: 63 - DNA, 6 bp, Artificial sequence
  Synthetic
gctcgc

SEQ ID NO: 64 - DNA, 6 bp, Artificial sequence
  Synthetic
tctcgc

SEQ ID NO: 65 - DNA, 5 bp, Artificial sequence
  Synthetic
gcgga

SEQ ID NO: 66 - DNA, 5 bp, Artificial sequence
  Synthetic
acgga

SEQ ID NO: 67 - DNA, 5 bp, Artificial sequence
  Synthetic
gcgga

SEQ ID NO: 68 - DNA, 5 bp, Artificial sequence
  Synthetic
acgga

SEQ ID NO: 69 - DNA, 7 bp, Artificial sequence
  Synthetic
atgcacc

SEQ ID NO: 70 - DNA, 7 bp, Artificial sequence
  Synthetic
ctgcacc

SEQ ID NO: 71 - DNA, 7 bp, Artificial sequence
  Synthetic
atgcacc

SEQ ID NO: 72 - DNA, 7 bp, Artificial sequence
  Synthetic
ctgcacc

SEQ ID NO: 73 - DNA, 9 bp, Artificial sequence
  Synthetic
tggaatgca

SEQ ID NO: 74 - DNA, 9 bp, Artificial sequence
  Synthetic
cggaatgca

SEQ ID NO: 75 - DNA, 9 bp, Artificial sequence
  Synthetic
tggaatgca

SEQ ID NO: 76 - DNA, 9 bp, Artificial sequence
  Synthetic
cggaatgca

SEQ ID NO: 77 - DNA, 7 bp, Artificial sequence
  Synthetic
caccaga

SEQ ID NO: 78 - DNA, 7 bp, Artificial sequence
  Synthetic
gaccaga

SEQ ID NO: 79 - DNA, 7 bp, Artificial sequence
  Synthetic
caccaga

SEQ ID NO: 80 - DNA, 7 bp, Artificial sequence
  Synthetic
gaccaga

SEQ ID NO: 81 - DNA, 6 bp, Artificial sequence
  Synthetic
ggcgca

SEQ ID NO: 82 - DNA, 6 bp, Artificial sequence
  Synthetic
agcgca

SEQ ID NO: 83 - DNA, 6 bp, Artificial sequence
  Synthetic
ggcgca

SEQ ID NO: 84 - DNA, 6 bp, Artificial sequence
  Synthetic
agcgca

SEQ ID NO: 85 - DNA, 10 bp, Artificial sequence
  Synthetic
atatacgccc

SEQ ID NO: 86 - DNA, 10 bp, Artificial sequence
  Synthetic
atatacgccc

SEQ ID NO: 87 - DNA, 10 bp, Artificial sequence
  Synthetic
atatacgccc

SEQ ID NO: 88 - DNA, 10 bp, Artificial sequence
  Synthetic
atatacgccc

SEQ ID NO: 89 - DNA, 12 bp, Artificial sequence
  Synthetic
gatcgcgctcgc

SEQ ID NO: 90 - DNA, 12 bp, Artificial sequence
  Synthetic
gatcgctctcgc

SEQ ID NO: 91 - DNA, 12 bp, Artificial sequence
  Synthetic
gatcgcgctcgc

SEQ ID NO: 92 - DNA, 12 bp, Artificial sequence
  Synthetic
gatcgctctcgc

SEQ ID NO: 93 - DNA, 10 bp, Artificial sequence
  Synthetic
gcggagcgga

SEQ ID NO: 94 - DNA, 10 bp, Artificial sequence
  Synthetic
gcggaacgga

SEQ ID NO: 95 - DNA, 10 bp, Artificial sequence
  Synthetic
gcggagcgga

SEQ ID NO: 96 - DNA, 10 bp, Artificial sequence
  Synthetic
gcggaacgga

SEQ ID NO: 97 - DNA, 15 bp, Artificial sequence
  Synthetic
acgagcaaatgcacc

SEQ ID NO: 98 - DNA, 15 bp, Artificial sequence
  Synthetic
acgagcaactgcacc

SEQ ID NO: 99 - DNA, 15 bp, Artificial sequence
  Synthetic
acgagcaaatgcacc

SEQ ID NO: 100 - DNA, 15 bp, Artificial sequence
  Synthetic
acgagcaactgcacc

SEQ ID NO: 101 - DNA, 17 bp, Artificial sequence
  Synthetic
gaggcaagtggaatgca

SEQ ID NO: 102 - DNA, 17 bp, Artificial sequence
  Synthetic
gaggcaagcggaatgca

SEQ ID NO: 103 - DNA, 17 bp, Artificial sequence
  Synthetic
gaggcaagtggaatgca

SEQ ID NO: 104 - DNA, 17 bp, Artificial sequence
  Synthetic
gaggcaagcggaatgca

SEQ ID NO: 105 - DNA, 14 bp, Artificial sequence
  Synthetic
tgcatgtcaccaga

SEQ ID NO: 106 - DNA, 14 bp, Artificial sequence
  Synthetic
tgcatgtgaccaga

SEQ ID NO: 107 - DNA, 14 bp, Artificial sequence
  Synthetic
tgcatgtcaccaga

SEQ ID NO: 108 - DNA, 14 bp, Artificial sequence
  Synthetic
tgcatgtgaccaga

SEQ ID NO: 109 - DNA, 12 bp, Artificial sequence
  Synthetic
tacgagggcgca

SEQ ID NO: 110 - DNA, 12 bp, Artificial sequence
  Synthetic
tacgagagcgca

SEQ ID NO: 111 - DNA, 12 bp, Artificial sequence
  Synthetic
tacgagggcgca

SEQ ID NO: 112 - DNA, 12 bp, Artificial sequence
  Synthetic
tacgagagcgca

SEQ ID NO: 113 - DNA, 19 bp, Artificial sequence
  Synthetic
ccggtaaggacgcgatcac

SEQ ID NO: 114 - DNA, 15 bp, Artificial sequence
  Synthetic
aggcggatgcgacca

SEQ ID NO: 115 - DNA, 20 bp, Artificial sequence
  Synthetic
aaaggcgtccgaggtgatac

SEQ ID NO: 116 - DNA, 19 bp, Artificial sequence
  Synthetic
gctacccgtgcgatgtgaa

SEQ ID NO: 117 - DNA, 17 bp, Artificial sequence
  Synthetic
cgggcaacaagctcgac

SEQ ID NO: 118 - DNA, 17 bp, Artificial sequence
  Synthetic
cggcgttcagcaagctc

SEQ ID NO: 119 - DNA, 20 bp, Artificial sequence
  Synthetic
cagaccttgcaggccgactc

SEQ ID NO: 120 - DNA, 95 bp, M. tuberculosis
gagctcgtatggcaccggaaccggtaaggacgcgatcaccagcggcatcgaggtcgtatggacgaacaccccgacgaaatgggacaacagtttcc

SEQ ID NO: 121 - DNA, 29 bp, Artificial sequence
  Synthetic
gtggtgatcggtaaggacgagatcaccac

SEQ ID NO: 122 - DNA, 29 bp, Artificial sequence
  Synthetic
ttggtgatcggtaaggacgagatcaccaa

SEQ ID NO: 123 - DNA, 29 bp, Artificial sequence
  Synthetic
atggtgatcggtaaggacgagatcaccat

SEQ ID NO: 124 - DNA, 29 bp, Artificial sequence
  Synthetic
ctggtgatcggtaaggacgagatcaccag